(19) United States
(12) Patent Application Publication
(10) Pub. No.: US 2025/0136667 A1
(43) Pub. Date: May 1, 2025

(54) ISOLATION OF THERAPEUTIC PROTEIN

(71) Applicant: Amgen Inc., Thousand Oaks, CA (US)

(72) Inventors: Qingchun ZHANG, Thousand Oaks, CA (US); Lidong HE, Thousand Oaks, CA (US); Mark KROENKE, Newbury Park, CA (US); Nicolas H. ANGELL, Newbury Park, CA (US); Suminda HAPUARACHCHI, Camarillo, CA (US)

(73) Assignee: Amgen Inc., Thousand Oaks, CA (US)

(21) Appl. No.: 18/681,252

(22) PCT Filed: Aug. 5, 2022
(86) PCT No.: PCT/US2022/074612

§ 371 Date: Feb. 5, 2024

Related U.S. Application Data
(60) Provisional application No. 63/230,483, filed on Aug. 6, 2021.

(51) Int. Cl. C07K 16/00; C07K 1/16

(52) U.S. Cl. CPC C07K 16/00; C07K 1/16

(57) ABSTRACT

Methods of isolating a therapeutic protein from a sample are described herein. Kits for isolating a therapeutic protein from a sample are described herein.

### CROSS-REFERENCE TO RELATED APPLICATIONS

This application claims the benefit of U.S. Provisional Patent Application No. 63/230,483, filed Aug. 6, 2021, the disclosure of which is incorporated by reference herein in its entirety.

### REFERENCE TO SEQUENCE LISTING

The present application is being filed along with a Sequence Listing in electronic format. The Sequence Listing is provided as a file entitled A-2883-WO01-SEC.xml created on Aug. 5, 2022, which is 4000 bytes in size. The information in the electronic format of the Sequence Listing is incorporated herein by reference in its entirety.

### FIELD

Embodiments herein relate to methods and kits for isolation of therapeutic protein from samples, such as ex vivo samples.

### BACKGROUND

Therapeutic monoclonal antibodies possess a wide variety of modifications (also referred to as attributes) associated with cell expression, manufacturing, and storage.[1] To assure pharmaceutical safety and efficacy, understanding how the critical quality attributes (CQAs) affect therapeutic protein quality is developed from the start of drug design.[2, 3] CQAs are controlled and monitored within defined intervals during manufacturing for consistent product quality.[4] CQAs evaluation was traditionally performed with in vitro studies. However, the acquired attribute knowledge may not represent the in vivo case due to lack of comparable physiological conditions. As a result, understanding the effect of attributes on drug metabolism with preclinical and clinical samples has gained growing interest.[5] Because serum contains proteins with various sizes and highly dynamic ranges of concentration which interfere with purification and downstream analysis of therapeutic proteins, in vivo study of attributes will involve purification of target therapeutic proteins from the serum matrix.

Immunoaffinity purification is generally applied to therapeutic antibody purification. Protein A-based chromatography has been utilized in majority of purification processes with the advantages of achieving high purity and recovery of therapeutic in one purification unit.[6, 7]

### SUMMARY

In some embodiments, a method of isolating a therapeutic protein from a sample is described. The therapeutic protein may comprise an IgG1 constant region comprising one or more of the following mutations numbered according to the EU system and selected from the group consisting of: L242C, A287C, R292C, N297G, V302C, L306C, and K334C. The method may comprise incubating the sample comprising the therapeutic protein with an antibody immobilized on a substrate. The antibody may bind selectively, compared to wild-type IgG1, to the IgG1 constant region comprising the one or more mutations. The immobilized antibody may thus bind to the IgG1 constant region of the therapeutic protein. The method may further comprise washing the immobilized antibody bound to the IgG1 constant region of the therapeutic protein. The method may further comprise eluting the therapeutic protein, thus isolating the therapeutic protein.

In any of the methods of isolating a therapeutic protein described herein, the sample may be an ex vivo sample of a human. In any of the methods of isolating a therapeutic protein described herein, the sample may comprise serum and/or serum proteins. In some methods, the ex vivo sample may comprise albumin bound to the therapeutic protein. In some methods, the ex vivo sample comprises immunoglobulins, in which the said immunoglobulins are different from the therapeutic protein.

In any of the methods of isolating a therapeutic protein described herein, the incubating is for about 15 minutes or less, such as 10 minutes or less.

In any of the methods of isolating a therapeutic protein described herein, the eluting may be at pH 3-3.5. In some methods, the elution is in a solution comprising acetic acid, optionally at 0.02% to 0.09% acetic acid, 0.02% to 0.07% acetic acid, 0.02% to 0.05% acetic acid, 0.05% to 0.09% acetic acid, or about 0.05% acetic acid.

In any of the methods of isolating a therapeutic protein described herein, the therapeutic protein may be an antigen binding protein and is bound to its antigen in the sample, in which the therapeutic protein remains bound to its antigen after the eluting.

In any of the methods of isolating a therapeutic protein described herein, the method may further comprise applying at least one analytical technique to the therapeutic protein.

In any of the methods of isolating a therapeutic protein described herein, the method may further comprise applying the eluted therapeutic protein to a chromatography column. In some methods, the chromatography may be size exclusion chromatography. In some methods, the therapeutic protein is an antigen binding protein bound to its antigen in the sample, wherein the therapeutic protein remains bound to its antigen after the elution, and the size exclusion chromatography comprises detecting a complex of the therapeutic protein bound to its antigen. By way of the example, the antigen binding protein may be a monoclonal antibody or an antibody protein product as described herein.

In any of the methods of isolating a therapeutic protein described herein, the method may further comprise performing mass spectrometry on the isolated antibody, such as LC-MS/MS.

In any of the methods of isolating a therapeutic protein described herein, the antibody (which may also be referred to as the “capture antibody”) may be a monoclonal antibody that binds specifically to the amino acid sequence CEEQYGSTYRC (SEQ ID NO: 1). In any of the methods of isolating a therapeutic protein described herein, the antibody (which may also be referred to as the “capture antibody”) may be a monoclonal antibody that was raised against a protein comprising the amino acid sequence ASTKGPSVFPLAPSSKSTSGGTAALGCLVKDYFPEPVTVSWNSGALTSGVHTFPAVLQSSGLYSLSSVVTVPSSSLGT QTYICNVNHKPSNTKVDKKVEPKSCDKTHTCPPCPAPELLGGPSVFLFPPKPKDTLMISRTPEVTCVVVDVSHEDPE VKFNWYVDGVEVHNAKTKPCEEQYGSTYRCVSVLTVLHQDWLNGKEYKCKVSNKALPAPIEKTISKAKGQPREPQ VYTLPPSREEMTKNQVSLTCLVKGFYPSDIAVEWESNGQPENNYKTTPPVLDSDGSFFLYSKLTVDKSRWQQGNVF SCSVMHEALHNHYTQKSLSLSPGK (SEQ ID NO: 2). In any of the methods of isolating a therapeutic protein described herein, the antibody (which may also be referred to as the “capture antibody”) may be a mouse monoclonal antibody.

In any of the methods of isolating a therapeutic protein described herein, the substrate may comprise a bead. In some methods, the bead comprises a non-porous monodisperse superparamagnetic bead, optionally wherein the bead is of a plurality of beads having an average diameter of about 2-4 μM, about 2-3 μM, about 2.5-3.5 μM, about 3 μM, or 2.8 μM.

In any of the methods of isolating a therapeutic protein described herein, the IgG1 constant region of the therapeutic protein comprises the mutations N297G and at least one of R292C and V302C. In any of the methods of isolating a therapeutic protein described herein, the IgG1 constant region of the therapeutic protein comprises the amino acid sequence: CEEQYGSTYRC (SEQ ID NO: 1) or

(SEQ ID NO: 2)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPCEEQYGSTYRCVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK.

In any of the methods of isolating a therapeutic protein described herein, the therapeutic protein is selected from the group consisting of an antibody such as a monoclonal antibody, an antigen-binding antibody fragment, an antibody protein product, a Bispecific T cell engager (BiTE®) molecule, optionally wherein the bi-specific T cell engager molecule comprises a half-life extension moiety, a bispecific antibody, a trispecific antibody, an Fc fusion protein, a recombinant protein, a recombinant virus, a recombinant T cell, a synthetic peptide, and an active fragment of a recombinant protein.

In some embodiments, a kit is described. The kit may comprise an antibody that binds selectively to an IgG1 constant region comprising the one or more mutations numbered according to the EU system and selected from the group consisting of: L242C, A287C, R292C, N297G, V302C, L306C, and K334C, as defined in any one of the methods of isolating a therapeutic protein described herein. The kit may further comprise a substrate. Additionally, regarding the kit, (i) the substrate may be configured for immobilization of the antibody thereon; or (ii) the antibody may be immobilized on the substrate.

### BRIEF DESCRIPTION OF THE DRAWINGS

FIG. 1 is a flow diagram illustrating a method for purifying a therapeutic protein according to some embodiments.

FIG. 2 is a SEC profile of mAb 1 standard and supernatant from mAb 1 incubation with capture antibody immobilized on DYNABEAD beads.

FIG. 3 is a SEC profile of mAb 1 after incubation with capture antibody immobilized on DYNABEAD beads for 10 min (dashed line), 1 hour (bold line), and overnight (unbolded line).

FIG. 4 is an SEC profile of SEFL2 immunoaffinity purified samples including mAb 1 spiked in PBS (unbolded line), mAb 1 spiked in human serum (bold line), and serum blank sample (flat line).

FIG. 5 is a SEC profile of mAb 1, mixture among mAb 1, antigen 1, and antigen 2 (2:1:1, bold line), and mixture between mAb 1 and antigen 2 (1:1, unbolded line). Consistent with drug-target complex model study in PBS buffer, slight excess of antigen 1 and antigen 2 led to a broad peak eluted ˜4.3 min, indicating a large complex among mAb 1, antigen 1, and antigen 2. And slight excess of antigen 2 led to peaks indicating antigen 2 bound mAb 1 with 1:1, 1:2, and 1:3 ratio.

FIG. 6 is a SE profile of SEFL2 immunoaffinity-purified antigen binding protein 1 spiked in PBS (bold line) and antigen binding protein 1 spiked in human serum (unbolded line).

### DETAILED DESCRIPTION

Described herein are methods useful for isolating a therapeutic protein from a sample, such as an ex vivo sample of a human. The therapeutic protein may comprise an IgG1 constant region comprising one or more mutations (e.g., one or more of L242C, A287C, R292C, N297G, V302C, L306C, and/or K334C, numbered according to the EU system). The sample comprising the therapeutic protein may be incubated with an antibody, immobilized on a substrate such as a bead. The antibody can bind selectively to the IgG1 constant region comprising the one or more mutations, compared to wild-type IgG1. The antibody bound to the therapeutic protein can be washed, and the therapeutic protein can then be eluted, so as to isolate the therapeutic protein. It is noted that such methods can be useful for isolating therapeutic proteins from ex vivo samples in order to identify molecular attributes of the therapeutic protein after it has been in an in vivo environment. It is contemplated that the methods may also be useful for isolating therapeutic proteins from other sample types, for example, in vitro samples or in process samples from manufacturing. Advantageously, such methods can isolate a therapeutic protein (such as an antibody) comprising an IgG1 constant region comprising the listed one or more mutations, but are agnostic to any binding target or targets of the therapeutic protein. Accordingly, generating critical reagents and the development of molecule specific affinity methods can be avoided. The methods described herein may be used for a number of different types of therapeutic proteins, for example monoclonal antibodies such as IgG monoclonal antibodies, bispecific T cell engager (BiTE®) molecules comprising a half-life extension (HLE) moiety (which may be referred to herein as an HLE-BiTE® molecule), hetero-IgGs, and IgGsc-Fv. Product quality attribute monitoring of individual molecules and further attribute understanding and characterization can be readily achieved without the development or use of molecule-specific affinity columns. This may include monitoring, understanding, and/or characterization of molecular attributes of therapeutic proteins in vivo (after administration to a subject), and recovered from ex vivo samples.

The methods described herein can separate a therapeutic protein from serum matrix and can be useful for understanding and monitoring molecular attributes (such as critical quality attributes) after therapeutic protein administration. Conventionally, there have been analytical challenges to isolating a therapeutic protein of interest from serum, including the low concentration of therapeutic proteins in serum and interference from high levels of endogenous proteins. As discussed in further detail herein, conventional affinity-based methods, such as protein A chromatography by targeting Fc regions of therapeutic antibodies, are generally not applicable due to a lack of selectivity and co-isolation with endogenous antibodies. Molecule-specific affinity chromatography methods have also been reported. However, protein-specific reagents or antibodies, which typically bind to complementarity-determining region (CDRs) of therapeutic proteins, were generated to achieve the desired selectivity in serum matrix. Development of such a molecule-specific method for each therapeutic protein is not only laborious and time-consuming, but also greatly limited by the availability of molecule specific reagents or antibody. By way of example, the methods described herein can isolate therapeutic proteins such as antibodies without a need to generate a capture antibody specific to their variable regions, and can isolate antibodies that comprise molecular attributes in their CDRs. Attributes in the CDRs, thought potentially relevant to efficacy and/or stability, may interfere with recovery of the antibody through conventional molecule-specific methods.

Immunoaffinity purification has conventionally been applied to therapeutic antibody purification. While Protein A-based chromatography has been utilized in majority of purification processes, human serum includes highly abundant polyclonal antibodies which are also captured by and coeluted with therapeutic antibodies from a Protein A column. Therefore, isolating a therapeutic protein (such as an antibody) of interest from other antibodies using Protein A chromatography can be challenging, as other antibodies may be co-purified along with an antibody of interest. Moreover, the abundant enzymatic peptides from these other antibodies' constant regions can suppress the signals of variable region peptides with lower abundance from therapeutic monoclonal antibodies using LC-MS/MS based approach after enzymatic digestion to monitor attributes in CDR/variable regions. In addition, the resulting complex peptide mixture can confound proteome analysis. For example, the origin of peptides cannot be assigned if more than one protein yielding identical enzymatic peptides are present.[8] As a result, the Protein A immunoaffinity purification method itself or in combination with LC-MS/MS is associated with challenges, and is typically not practicable.

As an alternative to Protein A chromatography, target ligand or customized anti-drug antibodies, binding the CDR region of target therapeutic protein, may be utilized to selectively pull the target therapeutic proteins from serum matrix before further attribute characterization.[9-13] This method generally features high sensitivity and high specificity, but involves the generation and use of customized anti-drug antibodies for each target therapeutic protein, which can require extensive development work. Moreover, attributes of interest in CDR regions (which may be particularly relevant to the efficacy of a therapeutic protein) could impact binding affinity to resins and lead to bias enrichment of certain populations. Furthermore, target ligand or customized anti-drug antibodies approaches may only purify the free form of therapeutic proteins in serum.[14]

Currently, many therapeutic proteins are engineered with mutations in an IgG constant region, which can eliminate undesired effector function. Therefore, an immunoaffinity approach targeting IgG1 constant regions comprising mutations (as described herein) of therapeutic proteins offers advantages of high selectivity (compared to Protein A column) and high versatility (applicable for all therapeutic proteins with an IgG1 constant region comprising these mutations). Methods as described herein have been used to isolate a hetero-IgG therapeutic protein and bispecific T cell engager (BiTE®) molecule comprising a half-life extension moiety, and followed by SEC characterizations. In certain embodiments, monoclonal antibody 1A3 is used to capture therapeutic proteins comprising an IgG1 comprising mutations R292C, N297G, and V302C (EU numbering). In certain embodiments, the antibody is immobilized on non-porous monodisperse superparamagnetic beads as described herein, for example DYNABEAD® beads. It has been observed that, compared to other beads such as SEPHAROSE® beads, DYNABEAD® beads exhibited higher reproducibility. It is noted that DYNABEAD® beads are generally uniform, and do not have an inner surface[15]. Without being limited by theory, it is contemplated that DYNABEAD® beads may be less prone to nonspecific binding. Accordingly, it is contemplated that methods described herein are useful for high selectivity and high versatility isolation of therapeutic proteins from samples such as ex vivo samples, and can facilitate the analysis of molecular attributes of the therapeutic proteins.

### Samples

The term “sample” and variations of this root term has its ordinary and customary meaning as would be understood by a person of ordinary skill in view of this disclosure. It refers to a composition that may contain a therapeutic protein a described herein, such as an ex vivo composition from a subject to whom the therapeutic protein has been administered. For example, a sample may comprise, consist essentially of, or consist of whole blood, plasma, serum, tissue biopsies, cerebrospinal fluid, peripheral blood mononuclear cells with in vitro stimulation, peripheral blood mononuclear cells, and lymphoid tissues. The sample may comprise, or may be expected to comprise the therapeutic protein. A “biological sample” will be understood herein to refer to a type of sample. In vitro or synthetic samples are also suitable for some embodiments, for example in process samples from the manufacturing of a therapeutic protein. The sample may be used in accordance with methods and/or kits as described herein. In some embodiments, the sample is an ex vivo sample of a human. In some embodiments, the sample comprises serum and/or serum proteins such as albumin. In various embodiments, the sample comprises albumin. The sample (e.g., an ex vivo sample of a human) may comprises albumin bound to the therapeutic protein. In some embodiments, the sample comprises immunoglobulins different from the therapeutic protein, for example immunoglobulins of a subject from whom the sample was obtained. Methods and kits described herein may be used to isolate the therapeutic protein from such immunoglobulins different from the therapeutic protein.

In some embodiments, the therapeutic protein is an antigen binding protein, antibody, Bi-specific T cell engager (BiTE®) molecule, bispecific antibody, trispecific antibody, or Fc fusion protein and the sample further comprises an antigen for the therapeutic protein. The therapeutic protein may be bound to its antigen in the sample.

In some embodiments, the sample comprises or consists of the therapeutic protein in a formulation. The formulation may be a pharmaceutically acceptable formulation. The formulation may comprise the biological therapy together with a pharmaceutically acceptable diluent, carrier, solubilizer, emulsifier, preservative, and/or adjuvant. Optionally, the sample may further comprise serum or serum protein.

Acceptable formulation materials for therapeutic proteins as described herein preferably are nontoxic to recipients at the dosages and concentrations employed. In certain embodiments, the pharmaceutical composition may contain formulation materials for modifying, maintaining or preserving, for example, the pH, osmolality, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, sucrose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. See, e.g., REMINGTON'S PHARMACEUTICAL SCIENCES, 18″ Edition, (A. R. Genrmo, ed.), 1990, Mack Publishing Company.

A suitable vehicle or carrier for the formulation may be water for injection, physiological saline solution or artificial cerebrospinal fluid, possibly supplemented with other materials common in compositions for parenteral administration. Neutral buffered saline or saline mixed with serum albumin are further exemplary vehicles. In specific embodiments, pharmaceutical compositions comprise Tris buffer of about pH 7.0-8.5, or acetate buffer of about pH 4.0-5.5, and may further include sorbitol or a suitable substitute therefor.

The formulation components are present preferably in concentrations that are acceptable to the site of administration. In certain embodiments, buffers are used to maintain the composition at physiological pH or at a slightly lower pH, typically within a pH range of from about 5 to about 8. For example, the pH of the formulation may be about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, or about 8.0.

### Therapeutic Proteins

As used herein “therapeutic protein,” and variations of this root term, has its ordinary and customary meaning as would be understood by one of ordinary skill in the art in view of this disclosure. It refers to a polypeptide for medical use in a subject, typically a human subject.

The therapeutic protein may comprise an IgG1 constant region comprising one or more of the following mutations numbered according to the EU system and selected from the group consisting of: L242C, A287C, R292C, N297G, V302C, L306C, and K334C. Unless stated otherwise, positions in (including positions of mutations in) constant regions will be referred to herein using EU numbering. By way of example, the mutation may comprise N297G and at least one of R292C and/or V302C. By way of example, the mutation may comprise R292C, N297G, and V302C. In some embodiments, the IgG1 constant region of the therapeutic protein comprises the amino acid sequence: CEEQYGSTYRC (SEQ ID NO: 1) or

(SEQ ID NO: 2)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPCEEQYGSTYRCVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK.

In methods described herein, the therapeutic protein may be selected from the group consisting of: an antibody (such as a monoclonal antibody, for example an IgG1 monoclonal antibody), an antigen binding protein, an antibody protein product, a Bi-specific T cell engager (BiTE®) molecule, a bispecific antibody, a trispecific antibody, an Fc fusion protein, a recombinant protein, a synthetic peptide, and an active fragment of a recombinant protein.

An “antibody” has its customary and ordinary meaning as understood by one of ordinary skill in the art in view of this disclosure. It refers to an immunoglobulin of with specific binding to the target antigen, and includes, for instance, chimeric, humanized, and fully human antibodies. By way of example, the antibody may be a monoclonal antibody. By way of example, human antibodies can be of a specified isotype, including IgG (including IgG1, IgG2, IgG3 and IgG4 subtypes), IgA (including IgA1 and IgA2 subtypes), IgM and IgE. A human IgG antibody generally comprises two full-length heavy chains and two full-length light chains. Antibodies may be derived solely from a single source, or may be “chimeric,” that is, different portions of the antibody may be derived from two or more different antibodies from the same or different species. It will be understood that once an antibody is obtained from a source, it may undergo further engineering, for example to enhance stability and folding. Accordingly, it will be understood that a “human” antibody may be obtained from a source, and may undergo further engineering, for example in the Fc region. The engineered antibody may still be referred to as a type of human antibody. Similarly, variants of a human antibody, for example those that have undergone affinity maturation, will also be understood to be “human antibodies” unless stated otherwise. In some embodiments, an antibody comprises, consists essentially of, or consists of a human, humanized, or chimeric monoclonal antibody. In various embodiments, the therapeutic protein comprises or consists of a chimeric, human, or humanized antibody comprising an IgG1 constant region comprising one or more of the following mutations numbered according to the EU system and selected from the group consisting of: L242C, A287C, R292C, N297G, V302C, L306C, and K334C. In various embodiments, the therapeutic protein is human or humanized antibody comprising an IgG1 constant region comprising one or more of the following mutations numbered according to the EU system and selected from the group consisting of: L242C, A287C, R292C, N297G, V302C, L306C, and K334C. In various embodiments, the therapeutic protein is a human antibody comprising an IgG1 constant region comprising one or more of the following mutations numbered according to the EU system and selected from the group consisting of: L242C, A287C, R292C, N297G, V302C, L306C, and K334C. By way of example, the mutation may comprise N297G and at least one of R292C and/or V302C according to EU numbering. By way of example, the mutation may comprise N297G, R292C, and V302C according to EU numbering.

A “heavy chain” of an antibody, antigen binding protein, antibody protein product, Bi-specific T cell engager molecule, bispecific antibody, or trispecific antibody includes a variable region (“VH”), and three constant regions: CH1, CH2, and CH3. A “light chain” of an antibody, antigen binding protein, antibody protein product, Bi-specific T cell engager molecule, bispecific antibody, or trispecific antibody includes a variable region (“VL”), and a constant region (“CL”). Human light chains include kappa chains and lambda chains. Example light chain constant regions suitable for antigen binding proteins include human lambda and human kappa constant regions.

In various aspects, the therapeutic protein is an antibody protein product. As used herein, the term “antibody protein product” refers to any one of several antibody alternatives which in various instances is based on the architecture of an antibody but is not found in nature. In some aspects, the antibody protein product has a molecular-weight within the range of at least about 12-150 kDa. In certain aspects, the antibody protein product has a valency (n) range from monomeric (n=1), to dimeric (n=2), to trimeric (n=3), to tetrameric (n=4), if not higher order valency. Antibody protein products in some aspects are those based on the full antibody structure and/or those that mimic antibody fragments which retain full antigen-binding capacity, e.g., scFvs, Fabs and VHH/VH (discussed below). The smallest antigen binding antibody fragment that retains its complete antigen binding site is the Fv fragment, which consists entirely of variable (V) regions. A soluble, flexible amino acid peptide linker is used to connect the V regions to a scFv (single chain fragment variable) fragment for stabilization of the molecule, or the constant (C) domains are added to the V regions to generate a Fab fragment [fragment, antigen-binding]. Both scFv and Fab fragments can be easily produced in host cells, e.g., prokaryotic host cells. Other antibody protein products include disulfide-bond stabilized scFv (ds-scFv), single chain Fab (scFab), as well as di- and multimeric antibody formats like dia-, tria- and tetra-bodies, or minibodies (miniAbs) that comprise different formats consisting of scFvs linked to oligomerization domains. The smallest fragments are VHH/VH of camelid heavy chain Abs as well as single domain Abs (sdAb). The building block that is most frequently used to create novel antibody formats is the single-chain variable (V)-domain antibody fragment (scFv), which comprises V domains from the heavy and light chain (VH and VL domain) linked by a peptide linker of ˜15 amino acid residues. A peptibody or peptide-Fc fusion is yet another antibody protein product. The structure of a peptibody consists of a biologically active peptide grafted onto an Fc domain. Peptibodies are well-described in the art. See, e.g., Shimamoto et al., mAbs 4(5): 586-591 (2012). Bispecific T-cell engage molecules, for example those comprising a half-life extension moiety are also examples of antibody protein products.

Therapeutic proteins suitable for the methods described herein can include polypeptides, including those that bind to one or more of the following: CD proteins, including CD3, CD4, CD8, CD19, CD20, CD22, CD30, and CD34; including those that interfere with receptor binding. HER receptor family proteins, including HER2, HER3, HER4, and the EGF receptor. Cell adhesion molecules, for example, LFA-I, Mol, pl50, 95, VLA-4, ICAM-I, VCAM, and alpha v/beta 3 integrin. Growth factors, such as vascular endothelial growth factor (“VEGF”), growth hormone, thyroid stimulating hormone, follicle stimulating hormone, luteinizing hormone, growth hormone releasing factor, parathyroid hormone, Mullerian-inhibiting substance, human macrophage inflammatory protein (MIP-1alpha), erythropoietin (EPO), nerve growth factor, such as NGF-beta, platelet-derived growth factor (PDGF), fibroblast growth factors, including, for instance, aFGF and bFGF, epidermal growth factor (EGF), transforming growth factors (TGF), including, among others, TGF-α and TGF-β, including TGF-β1, TGF-β2, TGF-β3, TGF-β4, or TGF-β5, insulin-like growth factors-I and -II (IGF-I and IGF-II), des(I-3)-IGF-I (brain IGF-I), and osteoinductive factors. Insulins and insulin-related proteins, including insulin, insulin A-chain, insulin B-chain, proinsulin, and insulin-like growth factor binding proteins. Coagulation and coagulation-related proteins, such as, among others, factor VIII, tissue factor, von Willebrand factor, protein C, alpha-1-antitrypsin, plasminogen activators, such as urokinase and tissue plasminogen activator (“t-PA”), bombazine, thrombin, and thrombopoietin; (vii) other blood and serum proteins, including but not limited to albumin, IgE, and blood group antigens. Colony stimulating factors and receptors thereof, including the following, among others, M-CSF, GM-CSF, and G-CSF, and receptors thereof, such as CSF-1 receptor (c-fms). Receptors and receptor-associated proteins, including, for example, flk2/flt3 receptor, obesity (OB) receptor, LDL receptor, growth hormone receptors, thrombopoietin receptors (“TPO-R,” “c-mpl”), glucagon receptors, interleukin receptors, interferon receptors, T-cell receptors, stem cell factor receptors, such as c-Kit, and other receptors. Receptor ligands, including, for example, OX40L, the ligand for the OX40 receptor. Neurotrophic factors, including bone-derived neurotrophic factor (BDNF) and neurotrophin-3, -4, -5, or -6 (NT-3, NT-4, NT-5, or NT-6). Relaxin A-chain, relaxin B-chain, and prorelaxin; interferons and interferon receptors, including for example, interferon-α, -β, and -γ, and their receptors. Interleukins and interleukin receptors, including IL-1 to IL-33 and IL-1 to IL-33 receptors, such as the IL-8 receptor, among others. Viral antigens, including an AIDS envelope viral antigen. Lipoproteins, calcitonin, glucagon, atrial natriuretic factor, lung surfactant, tumor necrosis factor-alpha and -beta, enkephalinase, RANTES (regulated on activation normally T-cell expressed and secreted), mouse gonadotropin-associated peptide, DNAse, inhibin, and activin. Integrin, protein A or D, rheumatoid factors, immunotoxins, bone morphogenetic protein (BMP), superoxide dismutase, surface membrane proteins, decay accelerating factor (DAF), HIV envelope, transport proteins, homing receptors, addressins, regulatory proteins, immunoadhesins, antibodies. Myostatins, TALL proteins, including TALL-I, amyloid proteins, including but not limited to amyloid-beta proteins, thymic stromal lymphopoietins (“TSLP”), RANK ligand (“RANKL” or “OPGL”), c-kit, TNF receptors, including TNF Receptor Type 1, TRAIL-R2, angiopoietins, and biologically active fragments or analogs or variants of any of the foregoing.

Examples of therapeutic proteins suitable for the methods described herein include antibodies or variants thereof comprising an IgG1 constant region comprising one or more of the following mutations numbered according to the EU system and selected from the group consisting of: L242C, A287C, R292C, N297G, V302C, L306C, and K334C, such as infliximab, bevacizumab, cetuximab, ranibizumab, palivizumab, abagovomab, abciximab, actoxumab, adalimumab, afelimomab, afutuzumab, alacizumab, alacizumab pegol, ald518, alemtuzumab, alirocumab, altumomab, amatuximab, anatumomab mafenatox, anrukinzumab, apolizumab, arcitumomab, aselizumab, altinumab, atlizumab, atorolimiumab, tocilizumab, bapineuzumab, basiliximab, bavituximab, bectumomab, belimumab, benralizumab, bertilimumab, besilesomab, bevacizumab, bezlotoxumab, biciromab, bivatuzumab, bivatuzumab mertansine, blinatumomab, blosozumab, brentuximab vedotin, briakinumab, brodalumab, canakinumab, cantuzumab mertansine, cantuzumab mertansine, caplacizumab, capromab pendetide, carlumab, catumaxomab, cc49, cedelizumab, certolizumab pegol, cetuximab, citatuzumab bogatox, cixutumumab, clazakizumab, clenoliximab, clivatuzumab tetraxetan, conatumumab, crenezumab, cr6261, dacetuzumab, daclizumab, dalotuzumab, daratumumab, demcizumab, denosumab, detumomab, dorlimomab aritox, drozitumab, duligotumab, dupilumab, ecromeximab, eculizumab, edobacomab, edrecolomab, efalizumab, efungumab, elotuzumab, elsilimomab, enavatuzumab, enlimomab pegol, enokizumab, enoticumab, ensituximab, epitumomab cituxetan, epratuzumab, erenumab, erlizumab, ertumaxomab, etaracizumab, etrolizumab, evolocumab, exbivirumab, fanolesomab, faralimomab, farletuzumab, fasinumab, fbta05, felvizumab, fezakinumab, ficlatuzumab, figitumumab, flanvotumab, fontolizumab, foralumab, foravirumab, fresolimumab, fulranumab, futuximab, galiximab, ganitumab, gantenerumab, gavilimomab, gemtuzumab ozogamicin, gevokizumab, girentuximab, glembatumumab vedotin, golimumab, gomiliximab, gs6624, ibalizumab, ibritumomab tiuxetan, icrucumab, igovomab, imciromab, imgatuzumab, inclacumab, indatuximab ravtansine, infliximab, intetumumab, inolimomab, inotuzumab ozogamicin, ipilimumab, iratumumab, itolizumab, ixekizumab, keliximab, labetuzumab, lebrikizumab, lemalesomab, lerdelimumab, lexatumumab, libivirumab, ligelizumab, lintuzumab, lirilumab, lorvotuzumab mertansine, lucatumumab, lumiliximab, mapatumumab, maslimomab, mavrilimumab, matuzumab, mepolizumab, metelimumab, milatuzumab, minretumomab, mitumomab, mogamulizumab, morolimumab, motavizumab, moxetumomab pasudotox, muromonab-cd3, nacolomab tafenatox, namilumab, naptumomab estafenatox, narnatumab, natalizumab, nebacumab, necitumumab, nerelimomab, nesvacumab, nimotuzumab, nivolumab, nofetumomab merpentan, ocaratuzumab, ocrelizumab, odulimomab, ofatumumab, olaratumab, olokizumab, omalizumab, onartuzumab, oportuzumab monatox, oregovomab, orticumab, otelixizumab, oxelumab, ozanezumab, ozoralizumab, pagibaximab, palivizumab, panitumumab, panobacumab, parsatuzumab, pascolizumab, pateclizumab, patritumab, pemtumomab, perakizumab, pertuzumab, pexelizumab, pidilizumab, pintumomab, placulumab, ponezumab, priliximab, pritumumab, PRO 140, quilizumab, racotumomab, radretumab, rafivirumab, ramucirumab, ranibizumab, raxibacumab, regavirumab, reslizumab, rilotumumab, rituximab, robatumumab, roledumab, romosozumab, rontalizumab, rovelizumab, ruplizumab, samalizumab, sarilumab, satumomab pendetide, secukinumab, sevirumab, sibrotuzumab, sifalimumab, siltuximab, simtuzumab, siplizumab, sirukumab, solanezumab, solitomab, sonepcizumab, sontuzumab, stamulumab, sulesomab, suvizumab, tabalumab, tacatuzumab tetraxetan, tadocizumab, talizumab, tanezumab, taplitumomab paptox, tefibazumab, telimomab aritox, tenatumomab, tefibazumab, teneliximab, teplizumab, teprotumumab, tezepelumab, TGN1412, tremelimumab, ticilimumab, tildrakizumab, tigatuzumab, TNX-650, tocilizumab, toralizumab, tositumomab, tralokinumab, trastuzumab, TRBS07, tregalizumab, tucotuzumab celmoleukin, tuvirumab, ublituximab, urelumab, urtoxazumab, ustekinumab, vapaliximab, vatelizumab, vedolizumab, veltuzumab, vepalimomab, vesencumab, visilizumab, volociximab, vorsetuzumab mafodotin, votumumab, zalutumumab, zanolimumab, zatuximab, ziralimumab, or zolimomab aritox.

In some embodiments, the therapeutic protein is a BiTE® molecule. BiTE® molecules are engineered bispecific antigen binding constructs which direct the cytotoxic activity of T cells against cancer cells. They are the fusion of two single-chain variable fragments (scFvs) of different antibodies, or amino acid sequences from four different genes, on a single peptide chain of about 55 kilodaltons. One of the scFvs binds to T cells via the CD3 receptor, and the other to a tumor cell via a tumor specific molecule. Blinatumomab (BLINCYTO® product) is an example of a BiTE® molecule, specific for CD19. BiTE® molecules that are modified, such as those modified to extend their half-lives, can also be used in the disclosed methods. In various aspects, the polypeptide is an antigen binding protein, e.g., a BiTE® molecule. In some embodiments, an antibody protein product comprises a BiTE® molecule.

Antibodies that Bind to IgG1 Constant Regions Comprising Mutations

Antibodies that bind to IgG1 constant regions comprising mutations as described herein may be used in methods and kits described herein. The antibody can bind specifically to an IgG1 comprising one or more mutations numbered according to the EU system and selected from the group consisting of: L242C, A287C, R292C, N297G, V302C, L306C, and K334C. For example, the IgG1 may comprise N297G and at least one of R292C and/or V302C according to EU numbering. The antibody may be a monoclonal antibody. By way of example, the antibody may be from a mammalian host organism, such as a mouse, hamster, rat, rabbit, goat, or donkey. For example, the antibody may be a mouse monoclonal antibody. The antibody may be provided immobilized on a substrate as described herein. The antibody immobilized on the substrate may also be referred to herein as a “capture antibody.” In some embodiments, the antibody binds to the amino acid sequence CEEQYGSTYRC (SEQ ID NO: 1). In some embodiments, the antibody comprises, consists essentially of, or consists of monoclonal antibody 1A3.

Suitable antibodies that bind to IgG1 constant regions comprising mutations as described herein may be prepared by techniques that are established in the art. For example, antibodies may be prepared by immunizing an animal (e.g., a mammal as described herein such as a mouse or rat or rabbit) with a protein comprising or consisting of the IgG1 constant region comprising mutation, and then by immortalizing spleen cells harvested from the animal after completion of the immunization schedule. The spleen cells can be immortalized using any technique known in the art, e.g., by fusing them with myeloma cells to produce hybridomas. See, for example, Antibodies; Harlow and Lane, Cold Spring Harbor Laboratory Press, 1^{st} Edition, e.g. from 1988, or 2^{nd} Edition, e.g. from 2014). By way of example, the antibody may be raised against a protein comprising or consisting of the amino acid sequence ASTKGPSVFPLAPSSKSTSGGTAALGCLVKDYFPEPVTVSWNSGALTSGVHTFPAVLQSSGLYSLSSVVTVPSSSLGT QTYICNVNHKPSNTKVDKKVEPKSCDKTHTCPPCPAPELLGGPSVFLFPPKPKDTLMISRTPEVTCVVVDVSHEDPE VKFNWYVDGVEVHNAKTKPCEEQYGSTYRCVSVLTVLHQDWLNGKEYKCKVSNKALPAPIEKTISKAKGQPREPQ VYTLPPSREEMTKNQVSLTCLVKGFYPSDIAVEWESNGQPENNYKTTPPVLDSDGSFFLYSKLTVDKSRWQQGNVF SCSVMHEALHNHYTQKSLSLSPGK (SEQ ID NO: 2). In some embodiments, the method comprising raising an antibody against a protein comprising or consisting of the amino acid sequence ASTKGPSVFPLAPSSKSTSGGTAALGCLVKDYFPEPVTVSWNSGALTSGVHTFPAVLQSSGLYSLSSVVTVPSSSLGT QTYICNVNHKPSNTKVDKKVEPKSCDKTHTCPPCPAPELLGGPSVFLFPPKPKDTLMISRTPEVTCVVVDVSHEDPE VKFNWYVDGVEVHNAKTKPCEEQYGSTYRCVSVLTVLHQDWLNGKEYKCKVSNKALPAPIEKTISKAKGQPREPQ VYTLPPSREEMTKNQVSLTCLVKGFYPSDIAVEWESNGQPENNYKTTPPVLDSDGSFFLYSKLTVDKSRWQQGNVF SCSVMHEALHNHYTQKSLSLSPGK (SEQ ID NO: 2). The antibody may be used to bind to IgG1 regions of therapeutic proteins in methods as described herein.

In certain embodiments, a B-cell that is producing a desired antibody is selected and the light chain and heavy chain variable regions are cloned from the B-cell according to established molecular biology techniques (WO 92/02551; U.S. Pat. No. 5,627,052; Babcook et al., Proc. Natl. Acad. Sci. USA 93:7843 48 (1996)) and described herein. B-cells from an immunized animal may be isolated from the spleen, lymph node, or peripheral blood sample by selecting a cell that is producing a desired antibody. B-cells may also be isolated from humans, for example, from a peripheral blood sample. Methods for detecting single B-cells that are producing an antibody with the desired specificity are well known in the art, for example, by plaque formation, fluorescence activated cell sorting, in vitro stimulation followed by detection of specific antibody, and the like. Methods for selection of specific antibody producing B-cells include, for example, preparing a single cell suspension of B-cells in soft agar that contains antigen. Binding of the specific antibody produced by the B-cell to the antigen results in the formation of a complex, which may be visible as an immunoprecipitate. After the B-cells producing the desired antibody are selected, the specific antibody genes may be cloned by isolating and amplifying DNA or mRNA according to methods known in the art.

An additional method for obtaining antibodies of the present disclosure is by phage display. See, e.g., Winter et al., 1994 Annu. Rev. Immunol. 12:433 55; Burton et al., 1994 Adv. Immunol. 57:191 280. Human or murine immunoglobulin variable region gene combinatorial libraries may be created in phage vectors that can be screened to select Ig fragments (Fab, Fv, sFv, or multimers thereof) that bind specifically to PCSK9 or variant or fragment thereof. See, e.g., U.S. Pat. No. 5,223,409; Huse et al., 1989 Science 246:1275-81; Sastry et al., Proc. Natl. Acad. Sci. USA 86:5728-32 (1989); Alting Mees et al., Strategies in Molecular Biology 3:1-9 (1990); Kang et al., 1991 Proc. Natl. Acad. Sci. USA 88:4363-66; Hoogenboom et al., 1992 J. Molec. Biol. 227:381-388; Schlebusch et al., 1997 Hybridoma 16:47-52; and references cited therein. For example, a library containing a plurality of polynucleotide sequences encoding Ig variable region fragments may be inserted into the genome of a filamentous bacteriophage, such as M13 or a variant thereof, in frame with the sequence encoding a phage coat protein. A fusion protein may be a fusion of the coat protein with the light chain variable region domain and/or with the heavy chain variable region domain. According to certain embodiments, immunoglobulin Fab fragments may also be displayed on a phage particle (see, e.g., U.S. Pat. No. 5,698,426).

Heavy and light chain immunoglobulin cDNA expression libraries may also be prepared in lambda phage, for example, using λImmunoZapTM(H) and λImmunoZapTM(L) vectors (Stratagene, La Jolla, California). Briefly, mRNA is isolated from a B-cell population, and used to create heavy and light chain immunoglobulin cDNA expression libraries in the λImmunoZap(H) and λImmunoZap(L) vectors. These vectors may be screened individually or co expressed to form Fab fragments or antibodies (see Huse et al., supra; see also Sastry et al., supra). Positive plaques may subsequently be converted to a non-lytic plasmid that allows high level expression of monoclonal antibody fragments from a microbial organism such as E. coli.

Once cells producing antibodies that bind to IgG1 comprising mutations according to the present disclosure have been obtained using any of the above-described immunization and other techniques, the specific antibody genes may be cloned by isolating and amplifying DNA or mRNA therefrom according to standard procedures as described herein. The antibodies produced therefrom may be sequenced and the CDRs identified and the DNA coding for the CDRs may be manipulated as described previously to generate other suitable antibodies that bind to IgG1 comprising mutations according to the present disclosure.

Molecular evolution of the complementarity determining regions (CDRs) in the center of the antibody binding site also has been used to isolate antibodies with increased affinity, as described by, for example, Schier et al., 1996, J. Mol. Biol. 263:551. Accordingly, such techniques are useful in preparing antibodies of the present disclosure.

### Substrates

In methods and kits described herein, an antibody that binds to an IgG1 comprising a mutation as described herein may be immobilized on a substrate, such as a bead. For example, the substrate may be a bead comprising or consisting of a non-porous monodisperse superparamagnetic bead (commercially available, for example, as DYNABEADS® beads). The beads may have an average diameter of about 2-4 μM, about 2-3 μM, about 2.5-3.5 μM, about 3 μM, or 2.8 μM. The antibody may be immobilized covalently on the bead via, for example, p-toluene-sulfonyl (tosylactivation) chemistry, or avidin-biotin chemistry. Optionally, the bead may comprise or consist of a Sepahrose bead. However, it has been observed that non-porous monodisperse superparamagnetic beads such as M-280 DYNABEADS® beads yield higher reproducibility than SEPHAROSE® beads.

### Methods of Isolating a Therapeutic Protein

In accordance with various embodiments described herein, methods of isolating a therapeutic protein from a sample are described. The therapeutic protein may comprise an IgG1 constant region comprising one or more mutations as described herein. The therapeutic protein may be incubated with an antibody that binds selectively to the IgG1 constant region of the therapeutic protein relative to wild-type IgG1. The antibody may be immobilized on a substrate as described herein. Thus, the immobilized antibody may bind the therapeutic protein. The immobilized antibody bound to the IgG1 constant region of the therapeutic protein may be washed. The therapeutic protein may be eluted, thus isolating the therapeutic protein. Optionally, at least one analytical technique is applied to the eluted (and isolated) therapeutic protein, for example to detect a presence and/or level of one or more molecular attributes of the therapeutic protein after it has been in an in vivo environment. For example, the analytical technique may comprise chromatography and/or mass spectrometry. In some embodiments, the IgG1 constant region of the therapeutic protein comprises one or more of the following mutations numbered according to the EU system and selected from the group consisting of: L242C, A287C, R292C, N297G, V302C, L306C, and K334C.

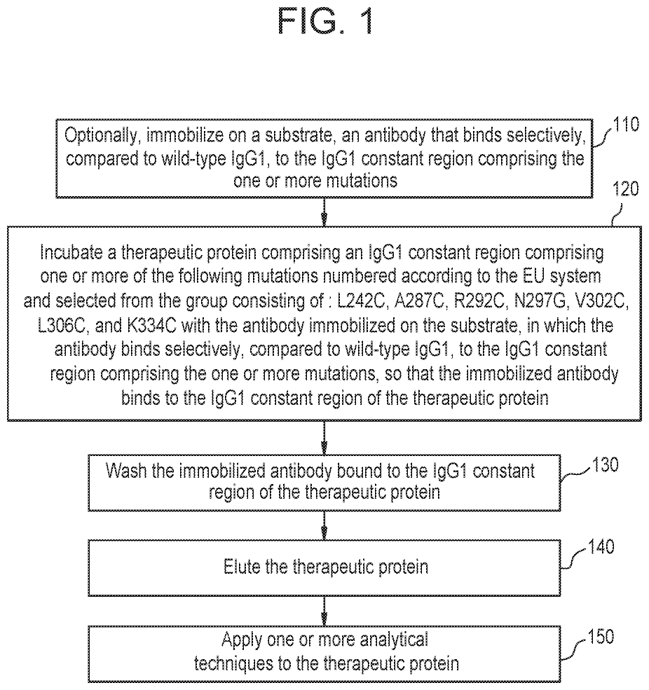

An exemplary method of isolating a therapeutic protein from a sample is illustrated in FIG. 1. Optionally, the method can comprise immobilizing on a substrate an antibody that binds selectively (compared to wild-type IgG1) to the IgG1 constant region comprising the one or more mutations as described herein 110. The method can further comprise incubating a therapeutic protein with the antibody immobilized on the substrate. The antibody can bind selectively (compared to wild-type IgG1) to the IgG1 constant region comprising the one or more mutations, so that the immobilized antibody binds to the IgG1 constant region of the therapeutic protein 120. As described herein, the therapeutic protein may comprise an IgG1 constant region comprising one or more of the following mutations numbered according to the EU system and selected from the group consisting of: L242C, A287C, R292C, N297G, V302C, L306C, and K334C. The method can comprise washing the immobilized antibody bound to the IgG1 constant region of the therapeutic protein 130. Optionally, the wash may be repeated one or more times. The therapeutic protein can be eluted 140. The therapeutic protein may be eluted in an acidic eluant. Acidic eluants, such as acetic acid, are described further herein. Thus, the therapeutic may be isolated. In some embodiments, one or more analytical techniques may be applied to the eluted therapeutic protein 150.

In some embodiments, the method further comprises immobilizing, on the substrate, the antibody that binds to the IgG1 constant region of the therapeutic protein. The immobilizing may comprise coupling the antibody to the substrate. For example, for a substrate that is a non-porous monodisperse superparamagnetic bead, the method can comprise covalently binding the antibody using tosylactivation chemistry, or biotin-avidin chemistry.

Incubation times of overnight, one hour, and 10 minutes were compared, and it was observed that the longer incubation times of overnight and one hour led to greater aggregation (Example 2). Accordingly, it is contemplated that incubation times of 20 minutes or less, such as about 10 minutes (or no more than 10 minutes) provide superior isolation of therapeutic proteins, while minimizing aggregation. In the method of some embodiments, the incubating is for about 20 minutes or less, for example, no more than 20, 15, 10, 8, 5, or 3 minutes. By way of example, the incubation may be performed on a roller.

After incubating the therapeutic protein with the antibody, the immobilized antibody bound to the IgG1 constant region of the therapeutic protein may be washed. The wash may be performed in a buffer, such as PBS. The wash may be at a pH of 6-8 or 7-8. Optionally, more than one wash may be performed, for example at least 1, 2, 3, 4, or 5 washes, including ranges between any two of the listed values, for example, 1-5 washes.

The incubated therapeutic protein may then be eluted. Different elution conditions for the therapeutic protein have been compared herein. Among elution solutions and pH's tested were 100 mM acetate buffer (pH 3.6, pH 4.6, and pH 5.6), acetic acid (0.005%, 0.01%, 0.05%, and 0.1%), 100 mM glycine (pH 3.0, pH 3.5, and pH 4.0), and Thermo gentle elution buffer. It was observed that 0.05% Acetic acid yielded less HMW peak area than the other elutions (Example 2). As the 0.05% acetic acid had a pH of 3.4 (compared to pH 3.9 for 0.005% acetic acid; pH 3.8 for 0.01% acetic acid; and pH 3.3 for 0.05% acetic acid) it was contemplated that an elution pH of about 3.4 to about 3.7 provides superior isolation while minimizing HWM species. As such, it will be appreciated that the elution may be in an acidic eluant such as acetic acid. In some embodiments, the elution is at a pH of 3.4 to 3.7, 3.4 to 3.6, 3.4 to 3.5, or about 3.4. In some embodiments, the elution is in a solution comprising acetic acid, for example 0.02% to 0.09% acetic acid, 0.02% to 0.07% acetic acid, 0.02% to 0.05% acetic acid, or 0.05% to 0.09% acetic acid. The elution in acetic acid may have a pH of 3.4 to 3.7, 3.4 to 3.6, 3.4 to 3.5, or about 3.4.

It may be desirable to isolate therapeutic protein that remains bound to its antigen from the sample. This may permit analyses informative of molecular attributes of the therapeutic protein that correlate with antigen binding. Accordingly, in some embodiments, the therapeutic protein is an antigen binding protein (e.g., antibody, Bi-specific T cell engager (BiTE®) molecule, bispecific antibody, or trispecific antibody) and is bound to its antigen in the sample. The therapeutic protein may remain bound to its antigen after the elution.

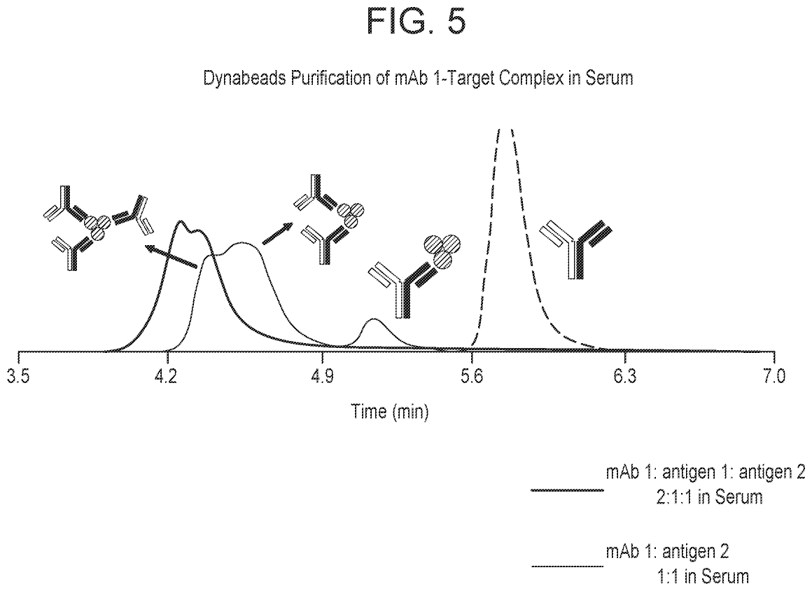

Following elution of the therapeutic protein, the method may further comprise applying one or more analytical techniques to the therapeutic protein. For example, the presence and/or levels of molecular attributes may be identified. The eluted therapeutic protein may be analyzed by chromatography, such as size exclusion chromatography (SEC). SEC may identify relative amounts of therapeutic protein unbound to any target, and therapeutic protein bound to target. By way of example, the fraction of therapeutic protein bound to target may be determined. By way of example, molecular attributes that correlate with the therapeutic protein being bound (or unbound) to target may be determined (See, e.g, PCT Pub. No. WO 2020/247790, which describes relationships between therapeutic protein complexes identified by SEC and molecular attributes, and which is incorporated by reference in its entirety herein). Also, stoichiometry of numbers of therapeutic protein and/or target in complex may be determined (See, e.g., FIG. 5). Accordingly, in some embodiments, the method comprises applying the eluted therapeutic protein to a chromatography column (e.g., an SEC column). For therapeutic proteins that are an antigen binding protein (such as an antibody or antibody protein product as described herein), the therapeutic protein may be bound to its antigen in the sample, the therapeutic protein may remain bound to its antigen after the elution, and the size exclusion chromatography may comprise detecting a complex of the therapeutic protein bound to its antigen

In some embodiments, at least one analytical technique is applied to the eluted therapeutic protein. Examples of suitable analytical techniques include mass spectrometry, chromatography, electrophoresis, spectroscopy, light obscuration, a particle method (such as nanoparticle/visible/micron-sized resonant mass or Brownian motion), analytical centrifugation, imaging or imaging characterization, or immunoassay. In some embodiments, the method comprises performing mass spectrometry on the isolated antibody. The mass spectrometry may be part of a peptide mapping analysis to identify the presence and/or levels of one or more molecular attributes. By way of example, peptide mapping by LC-MS/MS may be performed on the eluted therapeutic protein.

Examples of molecular attributes include acidic species, basic species, high molecular weight species, subvisible particle number, low molecular weight, middle molecular weight, glycosylation (such as non-glycosylated heavy chain or high mannose), non-heavy chain and light chain, deamidation, deamination, cyclization, oxidation, isomerization, fragmentation/clipping, N-terminal and C-terminal variants, reduced and partial species, folded structure, surface hydrophobicity, chemical modification, covalent bond, a C-terminal amino acid motif PARG, or a C-terminal amino acid motif PAR-Amide.

Upon isolation, the therapeutic protein may be free or substantially free of other proteins of the sample. For example, the isolated therapeutic protein may be at least one: (1) free of at least some other proteins with which it would normally be found, (2) essentially free of other proteins from the species of the subject from whom the sample was derived, and/or (3) separated from at least about 50 percent of polynucleotides, lipids, carbohydrates, or other materials of the sample. By way of example, serum protein produced by the subject from whom the sample was derived may comprise no more than 3%, 2%, 1%, or 0.1% of proteins that reside in the composition of the isolated therapeutic protein. By way of example, immunoglobulins produced by the subject from whom the sample was derived may comprise no more than 3%, 2%, 1%, or 0.1% of proteins that reside in the composition of the isolated therapeutic protein. In some embodiments, the isolated therapeutic protein constitutes at least about 5%, at least about 10%, at least about 25%, or at least about 50% of the composition in which it resides.

### Kits

In accordance with various embodiments described herein, kits for isolating a therapeutic protein from a sample are described. The kit can comprise an antibody that binds selectively to an IgG1 constant region comprising one or more mutations as described herein. For example, the IgG1 constant region may comprise one or more mutations selected from the group consisting of: L242C, A287C, R292C, N297G, V302C, L306C, and K334C. For example, the IgG1 may comprise N297G and at least one of R292C and/or V302C. In some embodiments, the kit further comprises a substrate. The substrate may be configured for immobilization of the antibody on the substrate, or the antibody of the kit may be immobilized on the substrate. For example, the substrate may comprise a non-porous monodisperse superparamagnetic bead as described herein.

Optionally, the kit may further comprise one or more of wash buffer and/or elution buffer. For example, the elution buffer may have a pH of about 3.4 to about 3.7, about 3.4, 3.4 to 3.7, 3.4 to 3.6, or 3.4 to 3.5. The elution buffer may comprise acetic acid, for example 0.02% to 0.09% acetic acid, 0.02% to 0.07% acetic acid, 0.02% to 0.05% acetic acid, or 0.05% to 0.09% acetic acid.

### EXAMPLES

### Example 1: Materials

Human serum was purchased from EMD Millipore (Billerica, MA, USA). All therapeutic proteins (mAb 1 and antigen binding protein 1) and antigen 1 were obtained from Amgen Inc. DYNABEADS® M-280 tosylactivated, PBS buffer, acetic acid (≥99.99%), monobasic sodium phosphate, dibasic sodium phosphate, sodium chloride, Tris solution, and recombinant antigen 2 protein were acquired from Thermo Fisher Scientific (Waltham, MA, USA). Ammonium sulphate and bovine serum albumin (BSA) were obtained from Sigma Aldrich (St. Louis, MO, USA). Therapeutic proteins tested were obtained from Amgen Inc., and included mAb 1 (a hetero IgG bispecific monoclonal antibody that binds to antigen 1 and antigen 2 simultaneously), and antigen binding protein 1 (a bispecific T cell engager (BiTE®) molecule comprising a half-life extension (HLE) moiety). Both therapeutic proteins comprise an IgG1 constant region comprising mutations R292C, N297G, and V302C (EU numbering), and in particular comprised the amino acid sequence CEEQYGSTYRC (SEQ ID NO: 1) comprising these mutations.

### Example 2: Isolation of Therapeutic Protein mAb 1

### Preparation of DYNABEAD® Beads Coupled with 1A3 mAb

Applicant-generated anti-SEFL2 mAb (1A3 mAb) was coupled to the M-280 tosylactivated DYNABEADS® according to the manufacturer's protocol. Briefly, 50 mg beads were washed with PBS buffer (pH 7.4), and 1 mg 1A3 mAb in 3 M ammonium sulphate were added to the washed beads for incubation on a roller overnight. After removal of supernatant, PBS (pH 7.4) with 0.5% (w/v) BSA was then applied to the beads for one hour to prevent non-specific binding. After washing, the beads were reconstituted in PBS Buffer (˜20 mg/mL) (FIG. 1).

### Purification of Therapeutic Protein

100 μL beads (20 μg maximum binding capacity for target protein) were added to therapeutic protein containing solution, and incubation was performed on a roller. The tube was then put on a magnet device for ˜1 min before carefully removing the supernatant. The collected beads were washed with PBS three times before target protein elution (FIG. 1).

### Size-Exclusion Chromatography

A Waters Acquity UPLC System (Milford, MA, USA) with Acquity UPLC BEH SEC column (200 Å, 1.7 μm, 4.6 mm×300 mm) was used for separation prior to detection. Mobile phase A, B, C, and D consisted of 500 mM monobasic sodium phosphate, 500 mM dibasic sodium phosphate, 1000 mM sodium chloride, and water, respectively. Samples (6 μg) were loaded onto the SEC column with mobile phase (7% A: 13% B: 25% C: 55% D) at a flow rate of 0.4 mL/min, and isocratic elution was then performed with the same mobile phase composition and flow rate over 12 min. The eluate was monitored by TUV detector at 220 nm and 280 nm with sampling rate of 20 points/second. Data were manually interpreted by use of Empower 3 software (Waters).

### Discussion of Immunoaffinity Purification Conditions

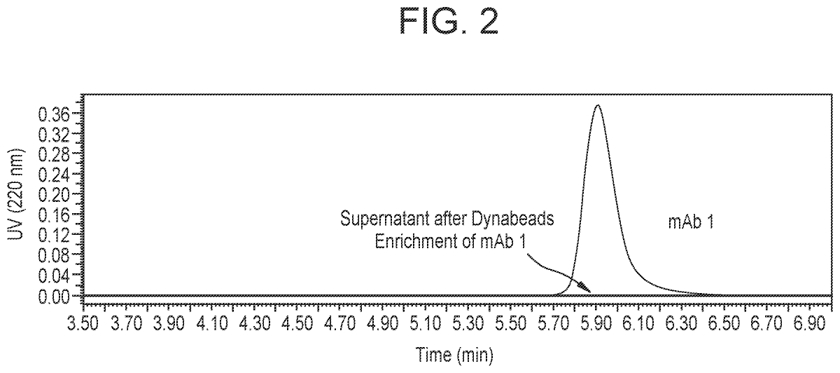

mAb 1 was used for immunoaffinity purification condition optimization. After mAb1 was incubated with the customized DYNABEAD® beads with 1A3, the supernatant was collected for SEC analysis. As shown in the SEC profile in FIG. 2, the supernatant did not generate UV signal, which indicates all mAb 1 were bound to the DYNABEAD® beads.

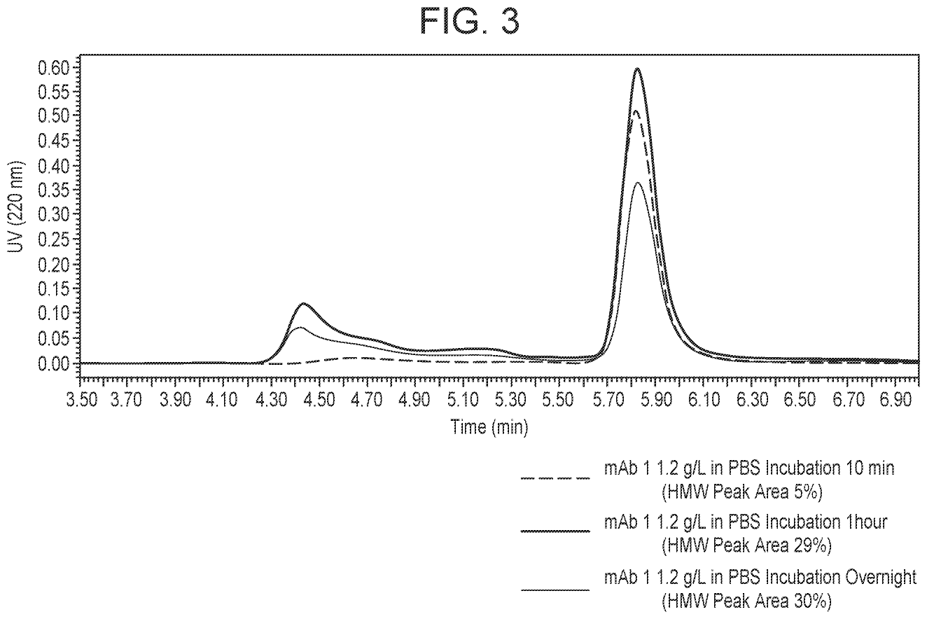

Next, incubation time was optimized by mixing mAb 1 with DYNABEADS® for 10 min, one hour, and overnight followed by DYNABEADS® wash and elution. Besides the main peak (corresponding to mAb 1 monomer) eluting at ˜5.8 min, an additional peak eluted at ˜4.5 min, corresponding to high molecular weight (HMW) species, was also observed in the SEC profile (FIG. 3). The HMW peak area was 5% for 10 min incubation time. However, incubation for one hour and overnight generated 29% and 30% HMW peak area, which indicates longer incubation time leads to high degree of protein aggregation. Therefore, incubation time was optimized to be 10 min.

The effect of elution solution on purified therapeutic protein was also studied. Applicant investigated different eluant with various pH including 100 mM acetate buffer (pH 3.6, pH 4.6, and pH 5.6), acetic acid (0.005%, 0.01%, 0.05%, and 0.1%), 100 mM glycine (pH 3.0, pH 3.5, and pH 4.0), and Thermo gentle elution buffer. 0.05% Acetic acid yielded minimum HMW peak area and was selected as the elution solution.

### Purification of Therapeutic Protein in Serum Matrix

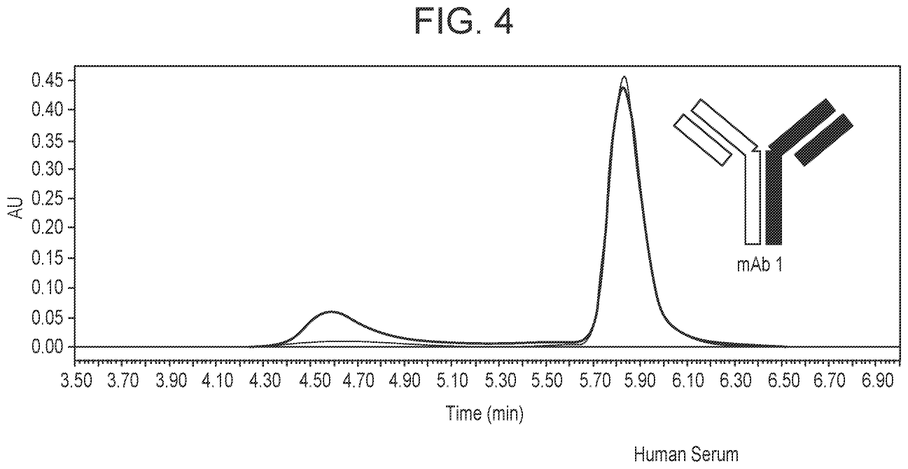

The optimized therapeutic protein purification procedure was applied to mAb 1 spiked in human serum (FIG. 4). The 1A3 mAb (coupled to the DYNABEADS®) specifically binds antibodies with SEFL2 mutation. As a result, blank serum sample yielded no interference peaks. mAb 1 was successfully purified from PBS and serum (shown as main peak). In addition, significant higher abundance of HMW region was detected in the sample of mAb 1 spiked in human serum. The HMW peak area percentage in serum matrix ranged from 55% to 36% (corresponding to mAb 1 concentration from 0.015 g/L to 1.2 g/L) and is much higher compared to that in PBS buffer matrix. Therefore, the high HMW peak area was potentially attributed to serum proteins binding to mAb 1 (or mAb 1 HMW species). The observed HMWs will be further characterized.

### Purification of Therapeutic Protein-Target Complex in Serum Matrix

To extend the application to immune complex purification, the developed therapeutic protein purification method was applied to human serum spiked with mAb 1 and its antigens (which may also be referred to as binding targets) (FIG. 5). mAb 1 is a bispecific hetero-IgG targeting both trimeric antigen 2 and antigen 1 simultaneously.

For human serum containing mAb 1 and antigen 2 spiked in with 1:1 ratio, two peaks at HMW elution window (unbolded line) were resolved, which indicates antigen 2 potentially binds to one mAb 1 and two or more mAb 1. For human serum containing mAb 1, antigen 1, and antigen 2 with 2:1:1 ratio, a broad HMW peak eluted early which indicates formation of a large complex between mAB 1 and its targets. All the peak assignments are solely based on retention time and have not been confirmed with other techniques.

### Example 3: Isolation of Therapeutic Protein “Antigen Binding Protein 1”

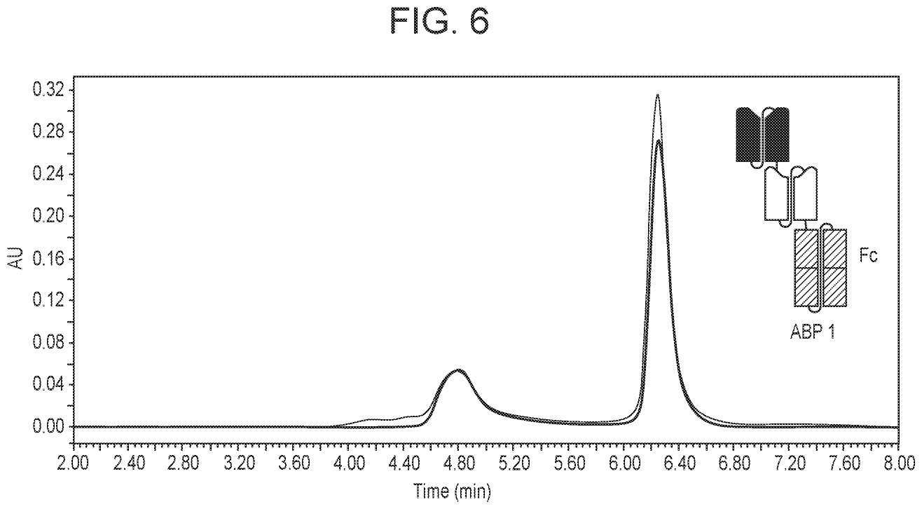

The approach described in Example 2 (and using materials as described in Example 1) was also applied to antigen binding protein 1, a bispecific T-cell engager (BiTE®) molecule with a half-life extension moiety as an example. As shown in FIG. 6, antigen binding protein 1 was successfully purified from PBS or serum matrix. The HMW peak is potentially caused by high density capturing antibody on the DYNABEADS®, and switching to Agarose beads with larger surface area may yield fewer HMW species.

### CONCLUSIONS

An immunoaffinity platform method useful for extracting therapeutic proteins from serum matrix was developed. In the method, the therapeutic proteins comprise specified mutations in the IgG1 region. IgG1 targeting antibody was covalently coupled to the magnetic beads for isolation and enrichment of target proteins. This platform approach has been successfully used to isolate mAb 1 (and its complex from serum) and antigen binding protein 1. The developed method is expected to be extended to other mAbs and modalities with the engineered mutations in IgG1. This method may be used to purify therapeutic proteins after in vivo exposure to aid CQA analysis.

### REFERENCES

Each of the following documents is incorporated by reference in its entirety herein:
1. Jefferis, R.: Posttranslational Modifications and the Immunogenicity of Biotherapeutics. J Immunol Res. 2016, U.S. Pat. No. 5,358,272 (2016)
2. Yu, L. X.: Pharmaceutical quality by design: product and process development, understanding, and control. Pharm Res. 25, 781-791 (2008)
3. Alt, N., Zhang, T. Y., Motchnik, P., Taticek, R., Quarmby, V., Schlothauer, T., Beck, H., Emrich, T., Harris, R. J.: Determination of critical quality attributes for monoclonal antibodies using quality by design principles. Biologicals. 44, 291-305 (2016)
4. Goetze, A. M., Schenauer, M. R., Flynn, G. C.: Assessing monoclonal antibody product quality attribute criticality through clinical studies. MAbs. 2, 500-507 (2010)
5. Li, Y., Huang, Y., Ferrant, J., Lyubarskaya, Y., Zhang, Y. E., Li, S. P., Wu, S. L.: Assessing in vivo dynamics of multiple quality attributes from a therapeutic IgG4 monoclonal antibody circulating in cynomolgus monkey. MAbs. 8, 961-968 (2016)
6. Moser, A. C., Hage, D. S.: Immunoaffinity chromatography: an introduction to applications and recent developments. Bioanalysis. 2, 769-790 (2010)
7. Liu, H. F., Ma, J., Winter, C., Bayer, R.: Recovery and purification process development for monoclonal antibody production. MAbs. 2, 480-499 (2010)
8. Mann, M., Kelleher, N. L.: Precision proteomics: the case for high resolution and high mass accuracy. Proc Natl Acad Sci USA. 105, 18132-18138 (2008)
9. Goetze, A. M., Liu, Y. D., Zhang, Z., Shah, B., Lee, E., Bondarenko, P. V., Flynn, G. C.: High-mannose glycans on the Fc region of therapeutic IgG antibodies increase serum clearance in humans. Glycobiology. 21, 949-959 (2011)
10. Zhang, Q., Schenauer, M. R., McCarter, J. D., Flynn, G. C.: IgG1 thioether bond formation in vivo. J Biol Chem. 288, 16371-16382 (2013)
11. Liu, Y. D., Chen, X., Enk, J. Z., Plant, M., Dillon, T. M., Flynn, G. C.: Human IgG2 antibody disulfide rearrangement in vivo. J Biol Chem. 283, 29266-29272 (2008)
12. Liu, Y. D., van Enk, J. Z., Flynn, G. C.: Human antibody Fc deamidation in vivo. Biologicals. 37, 313-322 (2009)
13. Geist, B. J., Davis, D., McIntosh, T., Yang, T. Y., Goldberg, K., Han, C., Pendley, C., Davis, H. M.: A novel approach for the simultaneous quantification of a therapeutic monoclonal antibody in serum produced from two distinct host cell lines. MAbs. 5, 150-161 (2013)
14. Neubert, H., Shuford, C. M., Olah, T. V., Garofolo, F., Schultz, G. A., Jones, B. R., Amaravadi, L., Laterza, O. F., Xu, K., Ackermann, B. L.: Protein Biomarker Quantification by Immunoaffinity Liquid Chromatography-Tandem Mass Spectrometry: Current State and Future Vision. Clin Chem. 66, 282-301 (2020)
15. Perform reproducible immunoprecipitation in less than 40 minutes. Accessible on the world wide web at assets.thermofisher.com/TFS-Assets/LSG/brochures/reproducible-immunoprecipitation-brochure.pdf.

1. A method of isolating a therapeutic protein from a sample, said therapeutic protein comprising an IgG1 constant region comprising one or more of the following mutations numbered according to the EU system and selected from the group consisting of: L242C, A287C, R292C, N297G, V302C, L306C, and K334C, the method comprising:
incubating the sample comprising the therapeutic protein with an antibody immobilized on a substrate, wherein the antibody binds selectively, compared to wild-type IgG1, to said IgG1 constant region comprising the one or more mutations, whereby the immobilized antibody binds to the IgG1 constant region of the therapeutic protein;
washing the immobilized antibody bound to the IgG1 constant region of the therapeutic protein; and
eluting the therapeutic protein, thereby isolating the therapeutic protein.

2. The method of claim 1, wherein the sample is an ex vivo sample of a human.

3. The method of claim 1, wherein the sample comprises serum and/or serum proteins.

4. The method of claim 3, wherein the ex vivo sample comprises albumin bound to the therapeutic protein.

5. The method of claim 2, wherein the ex vivo sample comprises immunoglobulins, wherein said immunoglobulins are different from the therapeutic protein.

6. The method of claim 1, wherein the incubating is for about 15 minutes or less.

7. The method of claim 1, wherein the eluting is at pH 3-3.5.

8. The method of claim 7, wherein the eluting is in a solution comprising 0.02% to 0.09% acetic acid.

9. The method of claim 1, wherein the therapeutic protein is an antigen binding protein and is bound to its antigen in the sample, and wherein the therapeutic protein remains bound to its antigen after the eluting.

10. The method of claim 1, further comprising applying at least one analytical technique to the therapeutic protein.

11. The method of claim 1, further comprising applying the eluted therapeutic protein to a chromatography column.

12. The method of claim 11, wherein the chromatography is size exclusion chromatography.

13. The method of claim 12, wherein the therapeutic protein is an antigen binding protein bound to its antigen in the sample, wherein the therapeutic protein remains bound to its antigen after the elution, and wherein the size exclusion chromatography comprises detecting a complex of the therapeutic protein bound to its antigen.

14. The method of claim 1, further comprising performing mass spectrometry on the isolated antibody.

15. The method of claim 1, wherein the antibody is a monoclonal antibody that binds specifically to the amino acid sequence CEEQYGSTYRC (SEQ ID NO: 1).

16. The method of claim 1, wherein the antibody is a monoclonal antibody that was raised against a protein comprising the amino acid sequence
(SEQ ID NO: 2)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPCEEQYGSTYRCVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK.

17. The method of claim 1, wherein the antibody is a mouse monoclonal antibody.

18. The method of claim 1, wherein the substrate comprises a bead.

19. The method of claim 18, wherein the bead comprises one or more non-porous monodisperse superparamagnetic beads, wherein each bead has an average diameter of about 2-4 μM.

20. The method of claim 1, wherein the IgG1 constant region of the therapeutic protein comprises the mutations N297G and at least one of R292C and V302C.

21. The method of claim 1, wherein the IgG1 constant region of the therapeutic protein comprises the amino acid sequence:
(SEQ ID NO: 1)
CEEQYGSTYRC
or
(SEQ ID NO: 2)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPCEEQYGSTYRCVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK.

22. The method of claim 1, wherein the therapeutic protein is selected from the group consisting of a monoclonal antibody, an antigen-binding antibody fragment, an antibody protein product, a bi-specific T cell engager (BiTE®) molecule, a bispecific antibody, a trispecific antibody, an Fc fusion protein, a recombinant protein, a recombinant virus, a recombinant T cell, a synthetic peptide, and an active fragment of a recombinant protein.

23. A kit comprising:
an antibody that binds selectively to an IgG1 constant region comprising the one or more mutations numbered according to the EU system and selected from the group consisting of: L242C, A287C, R292C, N297G, V302C, L306C, and K334C, and
a substrate, wherein:
(i) the substrate is configured for immobilization of the antibody thereon; or
(ii) the antibody is immobilized on the substrate.


SEQUENCE LISTING (2 sequences)

SEQ ID NO: 1 - protein, 11 aa, synthetic construct
  REGION 1..11: note = Engineered mmunoglobulin constant region sequence motif
CEEQYGSTYRC

SEQ ID NO: 2 - protein, 330 aa, synthetic construct
  REGION 1..330: note = Engineered immunoglobulin constant region sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK